(19) United States
(12) Patent Application Publication
(10) Pub. No.: US 2026/0256340 A1
(43) Pub. Date: Sep. 3, 2026

(54) MEDICAL SCOPE ACCESSORY

(71) Applicant: KEYMED (MEDICAL & INDUSTRIAL EQUIPMENT) LIMITED, Southend-on-Sea Essex (GB)

(72) Inventor: Andreas BAYER, Southend-on Sea Essex (GB)

(73) Assignee: KEYMED (MEDICAL & INDUSTRIAL EQUIPMENT) LIMITED, Southend-on-Sea Essex (GB)

(21) Appl. No.: 19/163,154

(22) PCT Filed: Feb. 15, 2024
(86) PCT No.: PCT/EP2024/053822

§ 371 Date: Sep. 8, 2025

Related U.S. Application Data
(60) Provisional application No. 63/450,757, filed on Mar. 8, 2023.

(30) Foreign Application Priority Data
Mar. 9, 2023 (GB) 2303433.3

(51) Int. Cl. A61B 1/00; A61B 1/31

(52) U.S. Cl. CPC A61B 1/00085; A61B 1/00101; A61B 1/31

(57) ABSTRACT

IN THE ABSTRACT:

device An accessory for use with a shaft of a medical scoping device. The accessory including a tubular body configured for application over a distal end of the medical scoping device shaft with the tubular body, in use, extending along a portion of the distal end of the shaft. The tubular body including a proximal end, a distal end, an inner surface, an outer surface, a cover defining the inner surface and the outer surface, and a stiffening member located between the inner surface and the outer surface. Where the cover is formed of a first material, the stiffening member is formed of a material harder than the first material, and the tubular body is radially expandable.

### CROSS-REFERENCE TO RELATED APPLICATION

The present application is based upon and claims the benefit of priority from PCT/EP 2024/053822, filed on Feb. 15, 2024, which claims priority to GB 2303433.3, filed on Mar. 9, 2023 and U.S. Provisional Application No. 63/450,757, filed on Mar. 8, 2023, the entire contents of each of which is incorporated herein by reference.

### BACKGROUND

### Field

The present disclosure relates to a device for use with a medical scope, such as an endoscope.

### Prior Art

US 2019/0183328 describes a cover for the shaft of a medical scoping device, in particular for fitting over the distal tip of an endoscope. The cover is a tubular body with proximal and distal ends and inner and outer circumferential surfaces. A plurality of projecting elements is provided on the outer surface of the cover which in use extend radially outward from the cover and facilitate the inspection of a body cavity or carrying out of other medical procedures with the medical scope.

However, it can be difficult to fit this type of cover to a shaft since the cover may buckle or become crumpled up or folded when inserting it on to the shaft.

### SUMMARY

The present disclosure provides an accessory for a shaft of a medical scoping device, the accessory comprising a tubular body configured for application over a distal end of the medical scoping device shaft with the tubular body, in use, extending along a portion of the distal end of the shaft, the tubular body comprising a proximal end, a distal end, an inner surface and the outer surface, the tubular body comprising a cover defining the inner surface and the outer surface, and a stiffening member located between the inner surface and the outer surface, wherein the cover is formed of a first, softer material and the stiffening member is formed of a second, harder material, and the tubular body is radially expandable. Such an accessory has greater rigidity, making it easier to fit the device on to a shaft. As the accessory is radially expandable it can be slightly expanded for fitting but can then grip tightly to the surface of the shaft. The inner and outer surfaces can be of a softer material to avoid damaging the shaft or body tissue.

### BRIEF DESCRIPTION OF THE DRAWINGS

The present disclosure refers, by way of example only, to the accompanying drawings in which:

FIG. 1 illustrates an exploded view and an assembled view of an accessory of the present disclosure;

FIG. 2 illustrates plan, side and bottom views, and perspective views from above and below, of the accessory of FIG. 1;

FIG. 3 illustrates plan, perspective and side views, and perspective views from above and below, of the cover of the accessory of FIG. 1;

FIG. 4 illustrates plan, side and bottom views, and perspective views from above and below, of the stiffening element of the accessory of FIG. 1;

FIGS. 5a to 5d illustrate side and perspective views of alternative embodiments of a stiffening element;

FIG. 6 illustrates end, side, plan and perspective views of a projecting element;

FIGS. 7a to 7c illustrate end and perspective views of alternative embodiments of a projecting element;

FIGS. 8a and 8b illustrate an accessory of the present disclosure during use.

### DETAILED DESCRIPTION

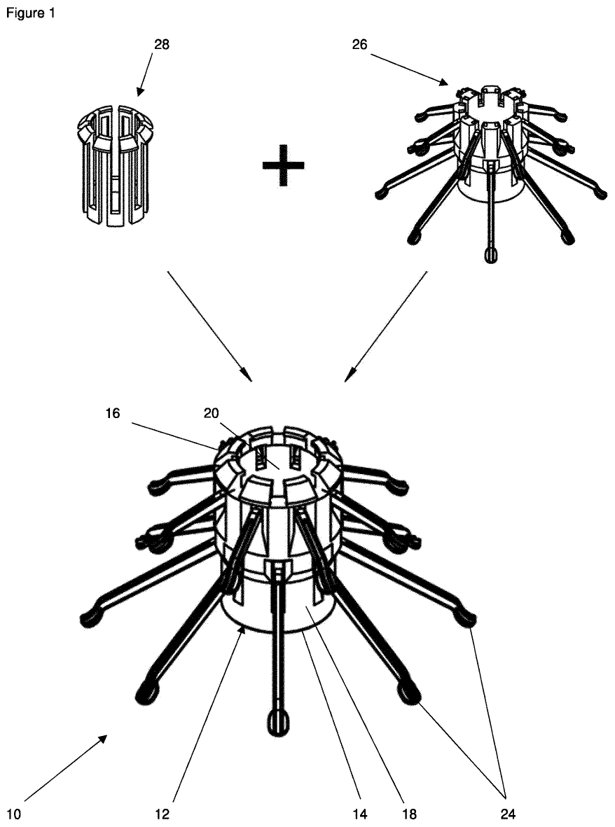

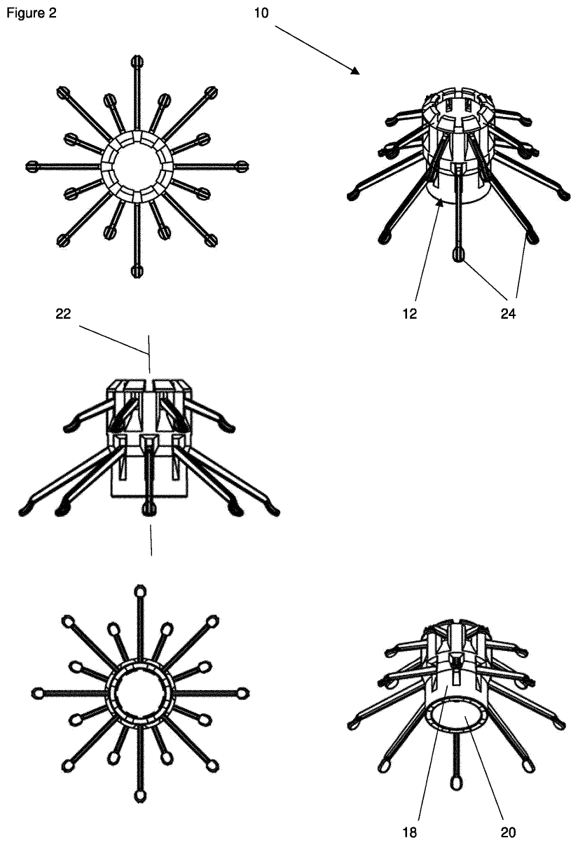

As shown in FIGS. 1 and 2, an accessory 10 for a shaft of a medical scoping device comprises a tubular body 12 which is open at each end. The tubular body 12 has a proximal end 14, a distal end 16, an outer circumferential surface 18 and an inner circumferential surface 20. The tubular body 12 has a longitudinal axis 22.

A plurality of projecting elements 24 may be provided to project radially outward from the outer surface 18 of the tubular body 12. As described further below, in the example shown in FIG. 1, two rows of eight projecting elements 24 are provided. The projecting elements 24 in each row are equally spaced around the circumference of the tubular body 12. However, there may be only one row or more than two rows. A different number of projecting elements 24 and a different spacing is also possible.

Each projecting element 24 may be able to move relative to the tubular body 12 in use, as described below. In a normal, rest position, each projecting element 24 projects outwardly from the tubular body 12 at an angle to the longitudinal axis 22, as shown in FIGS. 1 and 2.

The tubular body 12 is a two-part composite structure comprising a cover 26 and a stiffening member 28, shown separately at the top of FIG. 1. The tubular body 12 may be formed as a two-component injection moulded part, with the stiffening member 28 being moulded first and the cover 26 over moulded around it so that the stiffening member 28 becomes embedded within the cover 26. The cover 26 is formed of a first, softer material and the stiffening member 28 is formed of a second, harder material, but both parts are elastically deformable and are radially expandable, so that the tubular body 12 as a whole is radially expandable. The cover 26 and stiffening member 28 may be formed from a polymer material. The softer cover material forms both the inner surface 20 and the outer surface 18 of the tubular body 12. However, parts of the stiffening element 28 may be exposed as described further below.

In an alternative embodiment, the cover 26 may comprise a double skinned sleeve with an outer wall, an inner wall and an annular gap therebetween and the stiffening element 28 may locate within the gap. Openings may be provided in the cover 28 at the distal end to expose parts of the stiffening element 28.

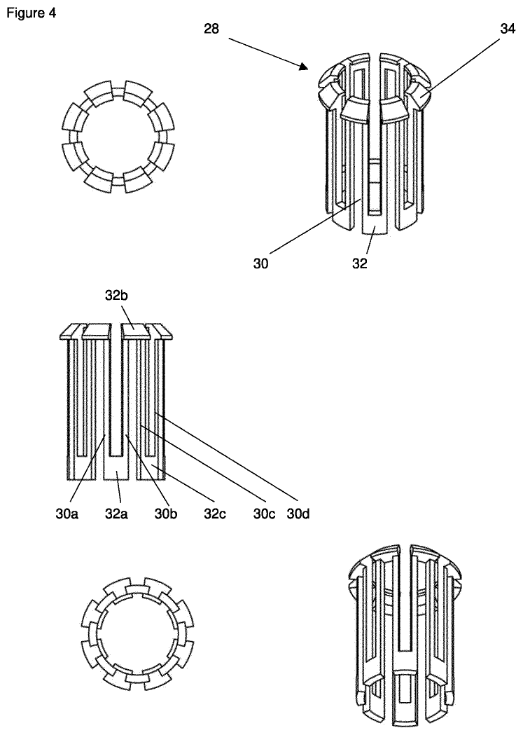

One embodiment of the stiffening member 28 is shown in FIG. 4. This comprises a generally cylindrical member which is formed by a series of axially extending legs 30 joined together by circumferentially extending struts 32 positioned between pairs of adjacent legs 30 and arranged alternately at the proximal and distal ends. That is, as illustrated in the side view in FIG. 4, a first leg 30a may be joined to a second leg 30b by a first strut 32a at the proximal end (that is at the lower end in the orientation shown in FIG. 4), the second leg 30b may be joined to a third leg 30c by a second strut 32b at the distal end (that is at the upper end in the orientation shown in FIG. 4), the third leg 30c may be joined to a fourth leg 30d by a third strut 32c at the proximal end and so on. Thus, in a plane perpendicular to the longitudinal axis, the stiffening element 28 is discontinuous and there are openings at intervals around the circumference. In this way, some radial expansion of the stiffening element 28 is possible by each pair of adjacent legs 30 diverging in a direction away from the strut 32 which connects them.

In the embodiment illustrated, the struts 32 formed at the distal end of the stiffening element 28 may be enlarged or provided with protrusions 34 which extend radially outwardly. In the assembled accessory 10, these protrusions 34 are left exposed and are not embedded within the cover 26. The exposed protrusions 34 together form a harder rim at the distal end 16 of the accessory 10.

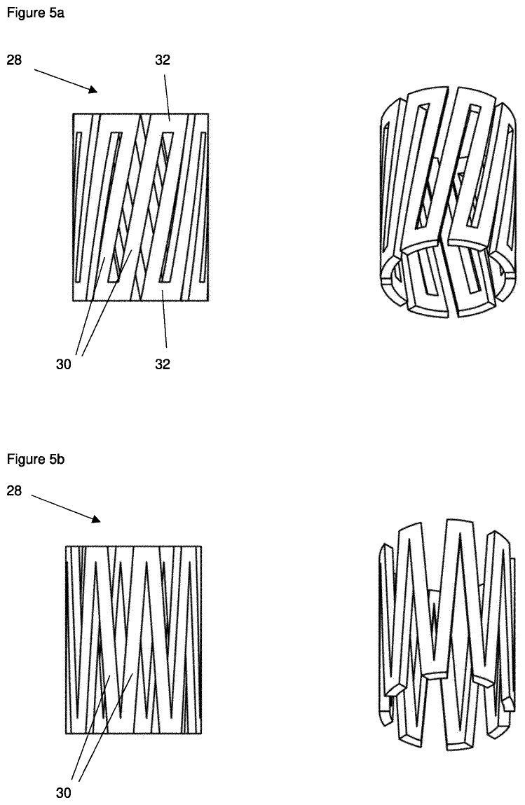

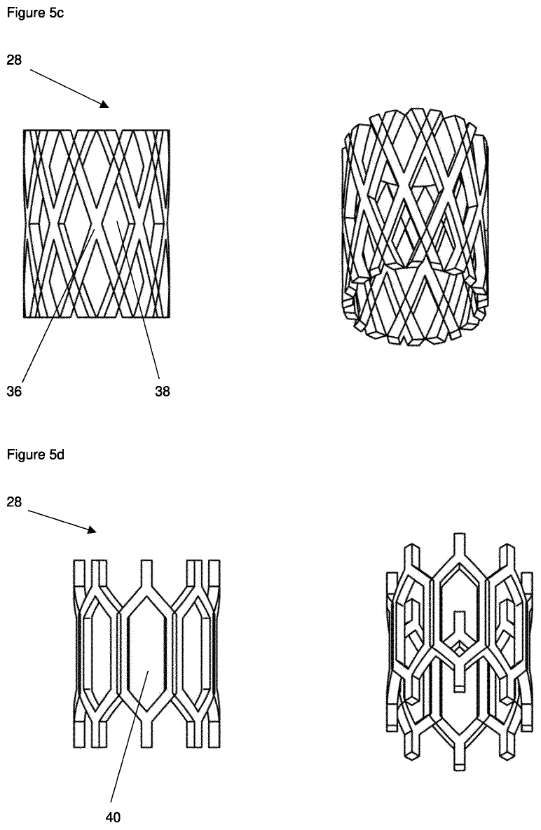

The precise configuration of the stiffening element 28 may be varied in a number of ways to provide a stiffening function whilst also permitting some radial expansion. For example, in the embodiment shown in FIG. 5a, the legs 30 are not parallel with the longitudinal axis 22 but are slanted at an angle relative to it.

In the embodiment of FIG. 5b, the legs 30 are arranged in a zig-zag formation with the legs 30 slanting alternately to form a series of interconnecting V-shapes. In this case, the connecting ends of adjacent legs 30 simply merge together.

Instead of legs 30 which are generally axially aligned and joined at alternate proximal and distal ends, the stiffening element 28 may be formed as a lattice structure. In the embodiment shown in FIG. 5c, diamond shaped cells 38 are created by a series of intersecting X-shaped legs 36. In the embodiment of FIG. 5d, a lattice with honeycomb or hexagonal shaped cells 40 is provided. In the embodiments of FIGS. 5c and 5d, at least one complete cell 38, 40 is provided around in the central portion of the stiffening element 28 whilst at the distal and proximal ends the cells 38, 40 are incomplete.

In all cases, the structure of the stiffening element 28 is such that in a plane perpendicular to the longitudinal axis the stiffening element 28 is discontinuous, to allow for radial expansion.

The stiffening element 28 of harder material provides rigidity to the accessory 10, making it easier to fit the accessory 10 on to the shaft 70 of a medical scoping device without the accessory 10 buckling or crumpling up. The softer material of the cover 26 forms in the inner and outer surfaces 18, 20, which will not damage the surface of the shaft 70 or body tissue.

As noted above, the tubular body 12 comprises a plurality of projecting arms 24 which, in use, help to flatten and stretch folds of tissue in order to assist with inspection and carrying out medical procedures. In the embodiment illustrated in FIGS. 1 to 3, the tubular body 12 comprises two rows 42, 44 of projecting arms 24, with eight arms 24 in each row. The arms 24 are equally spaced around the circumference. The first row 42 of arms 24 is joined to the tubular body 12 adjacent the distal end 16. The second row 44 is spaced axially from the first row 42 and is positioned generally in a central region of the tubular body 12.

The arms 24 in the first row 42 are shorter than those in the second row 44.

However, the arms 24 in all the rows may be the same length, or the arms 24 within one row may vary in length. Fewer or more rows of arms 24 may be provided with fewer or more arms 24 in each row.

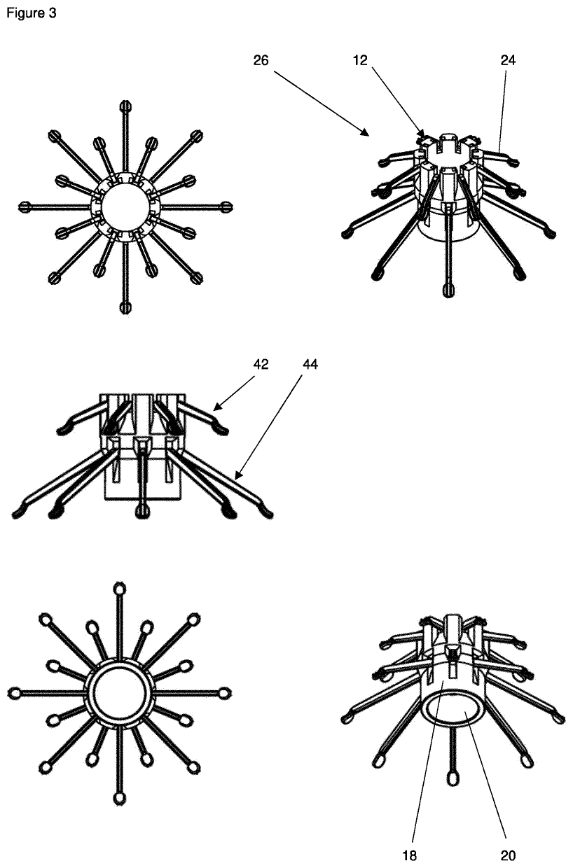

As seen in the plan views in FIGS. 2 and 3, the arms 24 in the first row 42 are staggered relative to the arms 24 in the second row 44 so that they do not overlap one another. However, it is also possible for the arms 24 in all the rows to be at the same orientation so that they do overlap one another. The arms 24 are formed of the same softer material as the cover 26 and are therefore able to flex when the accessory 10 is in use as discussed below.

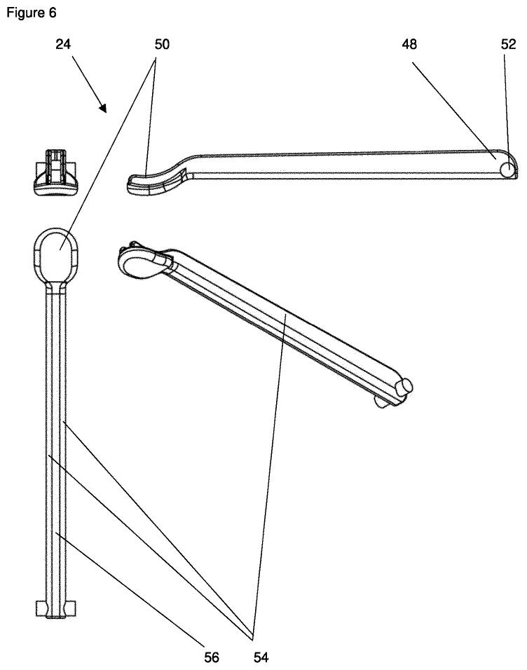

As best seen in FIG. 6, each arm 24 comprises a base 48 and a tip 50. The base 48 is connected to the tubular body 12 by connecting elements 52 which are sufficiently flexible to allow the arms 24 to pivot relative to the tubular body 12 in use. The connecting elements 52 for the shorter arms 24 may be smaller and weaker than the connecting elements 52 for the longer arms 24.

As shown in FIG. 6, in side profile, the arm narrows from the base 48 (shown on the right hand side in the Figure) towards the tip 50 (shown on the left hand side in the figure). In this orientation, the upper surface of the arm 24, which faces distally in use, may be provided with one or more ribs 54 extending along the arm from the base 48 to the tip 50. In the example shown, two ribs 54 are provided with a channel 56 therebetween. This reduces the surface area of the uppermost (distal facing) side of the arm 24 to reduce the area of the arm 24 making contact with the wall of the colon during insertion of the medical scoping device, in order to reduce friction. For the same reason, the uppermost (distal facing) surface should be smooth.

The lowermost or proximal facing side of the arm 24 may be roughened to increase resistance against the wall of the colon, in order to assist with gripping the tissue to flatten or straighten folds of tissue in use.

The tip 50 of each arm 24 may be enlarged and curved. In the side view, it can be seen that the tip 50 curves downwardly, that is in a proximal direction, compared to the rest of the arm 24, and then curves slightly upwardly again in order to create a cupped shape. In plan view, it can be seen that the tip 50 is widened with respect to the rest of the arm 24.

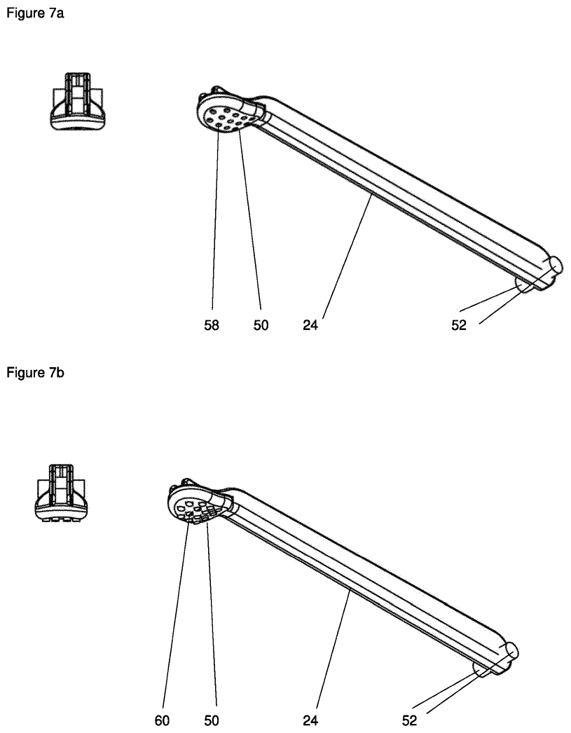

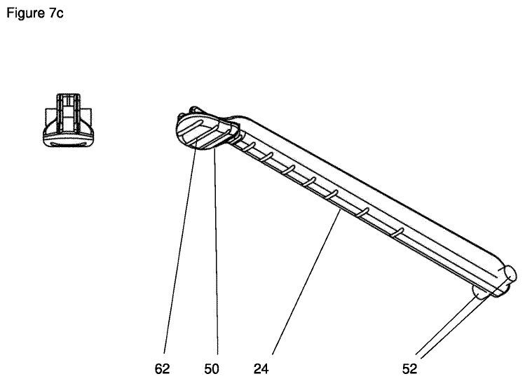

The lower, proximal facing side of the tip 50 may be provided with features to further enhance grip against the wall of the colon. This may be by way of a roughened surface texture or by the formation of an array of cavities 58 in the surface as shown in FIG. 7a, an array of protrusions 60 from the surface as shown in 7b, or a series of projecting ribs 62 as shown in 7c for example. As shown, the ribs 62 may also be formed along at least part of the arm 24 and not just at the tip 50. Similarly, the cavities 58 or protrusions 60 could also be formed along at least part of the arm 24 and not just at the tip 50.

In use, the accessory 10 is fitted over the distal end of a shaft 70 of a medical scoping device such as an endoscope. The shaft 70 of the medical scoping device may then be inserted into a patient's body, such as into the colon. Normally when examining the colon 72, the shaft 70 of the medical scoping device is advanced relatively quickly to the furthest point requiring examination and then gradually withdrawn more slowly with most of the visual examination carried out during the withdrawal.

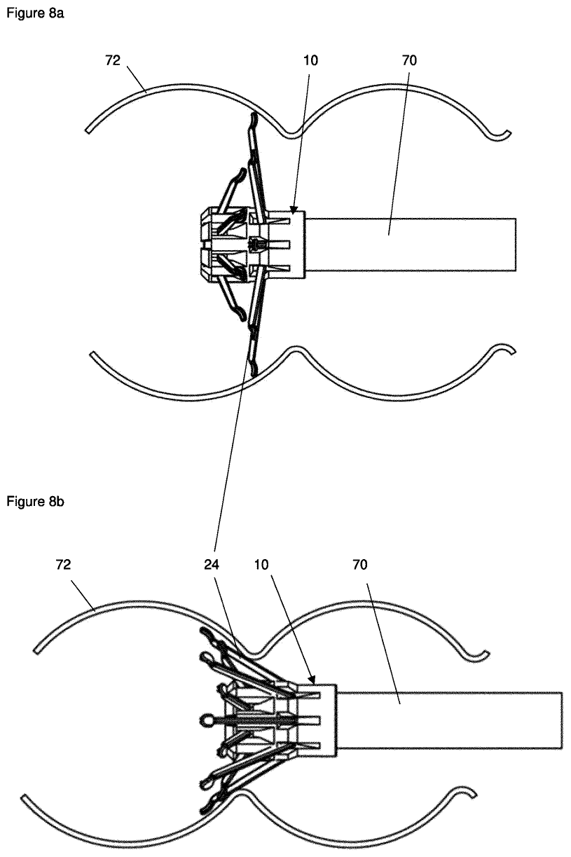

The arms 24 can flex relative to the tubular body 12 so that they do not inhibit insertion and withdrawal of the shaft 70. In the extended configuration as shown in FIG. 8a, the arms 24 are able to contact the wall of the colon 72. As the shaft 70 is withdrawn and the arm 24 encounter folds or narrower portions, the arms 24 contact and gently grip the tissue. Initially this may cause the arms 24 to be pushed inwardly towards the tubular body 12 as shown in FIG. 8b. Upon continued withdrawal of the shaft 70 the arms 24 tend to flex outwardly again and act to gently open and flatten colonic folds to assist with inspection. At the end of the procedure the shaft 70 is completely withdrawn from the patient's body. The cover 10 can then be removed from the shaft 70 for disposal, or for cleaning and re-use.

Thus, the present disclosure provides an improved accessory for the shaft of a medical scoping device which is easier to fit on to the shaft but remains flexible in use to assist with carrying out inspections and medical procedures.

While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

1. An accessory for use with a shaft of a medical scoping device, the accessory comprising:
a tubular body configured for application over a distal end of the medical scoping device shaft with the tubular body, in use, extending along a portion of the distal end of the shaft, the tubular body comprising:
a proximal end,
a distal end,
an inner surface,
an outer surface,
a cover defining the inner surface and the outer surface, and
a stiffening member located between the inner surface and the outer surface,
wherein the cover is formed of a first material and the stiffening member is formed of a second material harder than the first material, and
the tubular body is radially expandable.

2. The accessory as claimed in claim 1, wherein at least portions of the stiffening member are exposed from an interior of the cover.

3. The accessory as claimed in claim 2, wherein portions of the stiffening member at the distal end are exposed from the interior of the cover such that a rim at the distal end of the tubular body is harder than proximal portions of the tubular body.

4. The accessory as claimed in claim 1, wherein the stiffening member comprises a cylindrical member having a longitudinal axis and a plurality of openings formed such that the stiffening member is discontinuous in a plane perpendicular to the longitudinal axis.

5. The accessory as claimed in claim 4, wherein the stiffening member comprises a plurality of legs extending between the proximal and distal ends, wherein each leg is joined to an adjacent leg on one side by a connecting strut at the distal end and joined to an adjacent leg on an other side by a connecting strut at the proximal end.

6. The accessory as claimed in claim 4, wherein the stiffening member comprises a lattice formed by a plurality of interconnecting elements defining a plurality of cells therebetween.

7. The accessory as claimed in claim 6, wherein the cells are at least one of diamond shaped and hexagonal shaped.

8. The accessory as claimed in claim 1, wherein the tubular body further comprises a plurality of arms extending radially outwardly.

9. The accessory as claimed in claim 8, wherein each arm is joined to the tubular body by a flexible connecting element.

10. The accessory as claimed in claim 8, wherein the plurality of arms are arranged in at least two axially spaced rows.

11. The accessory as claimed in claim 10, wherein first arms of the plurality of arms arranged in a first row of the at least two axially spaced rows have a different length than second arms of the plurality of arms arranged in a second row of the at least two axially spaced rows.

12. The accessory as claimed in claim 8, wherein a distal facing surface of each of the plurality of arms has a smooth surface.

13. The accessory as claimed in claim 8, wherein a distal facing surface of each of the plurality of arms is formed with a pair of ribs extending from a base end of each of the plurality of arms to a free end of each of the plurality of arms and with a channel therebetween.

14. The accessory as claimed in claim 8, wherein at least part of a proximal facing surface of each of the plurality of arms is formed with a rough surface.

15. The accessory as claimed in claim 14, wherein at least part of the proximal facing surface of each of the plurality of arms is formed with one of a plurality of projections or a plurality of cavities.

16. The accessory as claimed claim 8, wherein a free end of each of the plurality of arms is formed with an enlarged tip relative to other portions of each of the plurality of arms.

17. The accessory as claimed in claim 16, wherein the enlarged tip is curved.
